(19) United States
(12) Patent Application Publication
(10) Pub. No.: US 2013/0078658 A1
(43) Pub. Date: Mar. 28, 2013

(54) METHOD OF QUANTIFYING RECOVERY RATE OF EXOSOME

(71) Applicants: Dong-hyun PARK, Chuncheon-si (KR); Hyun-jo KANG, Hwaseong-si (KR); Ko-bong CHOI, Yongin-si (KR); Myo-yong LEE, Suwon-si (KR)

(72) Inventors: Dong-hyun PARK, Chuncheon-si (KR); Hyun-jo KANG, Hwaseong-si (KR); Ko-bong CHOI, Yongin-si (KR); Myo-yong LEE, Suwon-si (KR)

(73) Assignee: SAMSUNG ELECTRONICS CO., LTD., Suwon-si (KR)

(21) Appl. No.: 13/491,258

(22) Filed: Jun. 7, 2012

(30) Foreign Application Priority Data
Sep. 23, 2011 (KR) 10-2011-0096373

(51) Int. Cl. G01N 21/76; G01N 21/64; C12N 9/96; C12P 21/00

(52) U.S. Cl. 435/8; 435/69.7; 435/188; 250/459.1

(57) ABSTRACT

A recombinant exosome comprising a fusion protein of a membrane protein and light-emitting protein, and a method of determining an exosome recovery rate by using the recombinant exosome are provided. Use of the method ensures accurate quantification of exosomes in a sample, and thus, improves the efficiency of an exosome-based diagnosis.

### CROSS-REFERENCE TO RELATED APPLICATIONS

This application claims the benefit of Korean Patent Application No. 10-2011-0096373, filed on Sep. 23, 2011, in the Korean Intellectual Property Office, the disclosure of which is incorporated herein by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 588 Byte ASCII (Text) file named “709792SequenceListing.txt,” created on Jun. 5, 2012.

### BACKGROUND

Exosomes are membrane-structured vesicles secreted by a wide range of cell types, and typically have a size of about 30 nm to about 100 nm in diameter. Studies using scanning electron microscopy (SEM) revealed that exosomes are not directly separated from plasma membranes; rather exosomes originate from specific intracellular regions called multivesicular bodies (MVBs). When MVBs fuse with the cell membrane, exosomes are released and secreted to the extracellular medium. A wide range of cell types, including red blood cells, tumor cells, and immune cells, such as B-lymphocytes, T-lymphocytes, dendritic cells (DCs), blood platelets, and macrophage, while alive, produce and secrete exosomes. Exosomes are known to be released from various different cell types both in normal and pathological conditions.

Exosomes also are known to include immunologically significant major histocompatibility complex (MHC) and heat shock proteins (HSP). Recent studies propose the use of exosomes as a vaccine composition that includes MHC class II proteins incorporated therein after isolation of the exosomes from a cell culture obtained by injection of genes able to induce expression of the HMC class II proteins into cancer cell lines (see KR 10-2009-47290A).

The presence of various types of exosomal microRNAs and a disease diagnostic method based on the presence or absence of the exosomal microRNAs, and the amount thereof, also have been disclosed (see KR 10-2010-0127768A). WO2009-015357A discloses a method of predicting association with a particular disease and a diagnostic method based on exosomal microRNA variations in a cancer-patient sample (e.g., blood, saliva, or tear drops) such as an increase or decrease in exosomal microRNA relative to a control group. Association of a particular exosomal microRNA isolated from a patient suffering from a particular disease (lung disease) with the disease that was found via exosomal analysis has been disclosed in detail. Other diagnostic methods for kidney diseases using exosomal proteins are currently being researched.

For accurate exosome-based diagnosis, accurate quantification of exosomes in a patient is very crucial. Measurement of a quantitative difference between experimentally recovered exosomes and actual exosomes present in a sample is important for higher-accuracy exosomal diagnosis. That is, measurement of exosome recovery rate after isolation of the exosomes is crucial in exosome-based diagnosis. Presently available exosomal quantification methods typically rely on specific antibody-antigen immunoreaction. However, such a method cannot be used when there are antigens in common between artificially manipulated exosomes and naturally occurring exosomes. Furthermore, the use of antibodies may complicate the overall quantification method.

Therefore, there remains a need for additional high-accuracy exosomal quantification methods and exosome-based diagnostic methods.

### SUMMARY

The invention provides a method of determining an exosome recovery rate comprising (a) mixing (i) a sample comprising exosomes with and (ii) a known amount of recombinant exosomes that comprise a fusion protein of a membrane protein and a light-emitting protein to obtain a mixture; (b) isolating the exosomes from the mixture; (c) detecting the amount of the recombinant exosomes among the isolated exosomes; and (d) determining the exosome recovery rate based on a ratio of the amount of recombinant exosomes after the separation to the known amount of recombinant exosomes mixed with the sample before the separation.

The invention also provides a recombinant exosome comprising a fusion protein, wherein the fusion protein comprises a membrane protein and a light-emitting protein. In a related aspect, the invention provides a method of preparing a recombinant exosome comprising (a) administering an expression vector to a cell, wherein the expression vector encodes a fusion protein comprising a membrane protein and a light-emitting protein, and (b) isolating the recombinant exosome from the cell.

### BRIEF DESCRIPTION OF THE DRAWINGS

These and/or other aspects will become apparent and more readily appreciated from the following description of the embodiments, taken in conjunction with the accompanying drawings in which:

FIG. 1 is a diagram of a recombinant vector to be introduced into cells for transfection in constructing a recombinant exosome according to an embodiment of the present disclosure.

FIG. 2 is a diagram of a recombinant exosome preparing method according to an embodiment of the present disclosure.

FIG. 3 is a diagram of a method of determining an exosome recovery rate using the recombinant exosome, according to an embodiment of the present disclosure.

FIG. 4 is a graph that illustrates the exosomal targeting efficiency of the fusion proteins of the recombinant exosomes. The fold fluorescence is indicated on the y-axis and the fusion proteins are indicated on the x-axis.

FIG. 5 is a photograph of a Western blot illustrating cellular expression of a fusion protein including a membrane protein (EpCAM) and fluorescent protein (EGFP) and a fusion protein of the membrane protein (EpCAM) and luciferase (RLUC).

FIG. 6 is a graph of cellular expression of fusion proteins and exosomal targeting efficiency with respect to types of membrane proteins in recombinant vectors, after transfection into cell line (via measuring fluorescence). Fluorescence (×100) is indicated on the y-axis and the particular fusion proteins are indicated on the x-axis.

FIG. 7 is a graph of cellular expression of fusion proteins and exosomal targeting efficiency with respect to types of membrane proteins in recombinant vectors after transfection into cell line (via measuring luminance). Luminance is indicated on y-axis and the particular fusion proteins are indicated on the x-axis.

FIG. 8 is a graph illustrating correlation between total exosomal protein and fluorescence when a CD63-GFP fusion protein was used, indicating that a minimum detectable amount of exosomes is about 25 ng. Fluorescence is indicated on the y-axis and total exosomal protein (ng) is indicated on the x-axis.

FIG. 9 is a graph illustrating proportional correlation between total exosomal protein (recombinant exosome including a EpCAM-RLUC fusion protein) and luminance, indicating that a minimal detectable amount of exosomes is about 80 ng. Luminance is indicated on the y-axis and total exosomal protein (μg) is indicated on the x-axis.

FIG. 10 is a graph with the activity ratio in luminance after exosome lysis to before exosome lysis (ratio of lysis/non-lysis) on the y-axis and the particular fusion proteins for the exosomes on the x-axis.

FIG. 11 is a graph illustrating results of quantifying exosomes in samples with or without consideration of recovery rate. The exosome amount is indicated on the y-axis for each of the reactions indicated on the x-axis.

### DETAILED DESCRIPTION

Provided herein is a method of determining an exosome recovery rate using recombinant exosomes that comprise a fusion protein of a membrane protein and a light-emitting protein. In particular, the inventive method comprises(a) mixing a sample comprising exosomes with a known quantity of the recombinant exosomes to obtain a mixture; (b) isolating the exosomes from the mixture; (c) detecting the amount of the recombinant exosomes among the isolated exosomes; and (d) determining an exosome recovery rate based on a ratio of the amount of recombinant exosomes after the separation to the known amount of recombinant exosomes mixed with the exosome-containing sample before the separation.

As used herein, the term “membrane protein” refers to a protein or glycoprotein that can reside in a liquid bilayer of a cell membrane. Membrane proteins include any proteins which can penetrate the lipid bilayer, or which can reside on a surface layer of the cell membrane, for example, receptors of enzymes, peptide hormones, and local hormones, sugar acceptors/carriers, and cell membrane antigens.

The membrane protein can be any protein or fragment thereof that penetrates a lipid bilayer. In one embodiment, the membrane protein is a cellular membrane protein, for example, an exosomal membrane protein. The membrane protein can be a portion or fragment of a full-length membrane protein sufficient to introduce the fusion protein into the exosomes, particularly the lipid membrane of the exosome. For example, the membrane protein can comprise an N-terminus or C-terminus region of the membrane protein (e.g., about 5 or more, about 10 or more, about 15 or more, about 20 or more, or about 25 or more contiguous amino acids from the C-terminus or N-terminus of a full-length membrane protein). Examples of membrane proteins include EpCAM, Hsc70, MHC I, Tsg101, calnexin, gp96, CD63, CD81, and L1.

As used herein, the term “light-emitting protein” refers to any protein that is able to emit light by a change in physical conditions or by a chemical process. The light-emitting protein can be, for example, a fluorescent protein, a photoprotein, or a luciferase. Examples of fluorescent proteins include, but are not limited to, a green fluorescent protein (GFP), a yellow fluorescent protein (YFP), and a red fluorescent protein (REP).

The light-emitting protein can be positioned inside or outside the exosomes. The position of the light-emitting protein relative to the exosome will depend upon the orientation of the particular membrane protein used, and the position of the light-emitting protein relative to the membrane protein in the fusion protein construct. For example, when a membrane protein is used that positions itself in the exosomal wall with its C-terminus towards the interior of the exosome, and the light-emitting protein is linked (directly or indirectly by a linker) to the C-terminus of the membrane protein, the light-emitting protein can be located inside the exosome. Similarly, if the light-emitting protein is linked (directly or indirectly by a linker) to the N-terminus of such a membrane protein, the light-emitting protein can be positioned outside of the exosome. The opposite is true when using a membrane protein that orients itself in the exosome wall with its C-terminus towards the outside of the exosome, and the N-terminus towards the interior of the exosome.

mixing an exosome-containing sample and a recombinant exosome that includes a fusion protein of a membrane protein and a light-emitting protein that are linked togetherThe membrane protein and light-emitting protein that comprise the fusion protein (e.g., directly or via a linker). For example, the light-emitting protein can be directly linked to the membrane protein (e.g., an N-terminus or C-terminus of the membrane protein).

As used herein, the term “linker” refers to a peptide that is able to link the light-emitting protein and membrane protein together. The linker can be any suitable length, such as about 1 to about 50 (e.g., 5, 10, 15, 20, 25, 30, 35, 40, or 55) amino acids. In a preferred embodiment, the linker comprises about 5 to about 20 amino acids.

The sample containing the exosomes (i.e., the exosome-containing sample) can be any suitable sample containing exosomes (e.g., naturally occurring exosomes). In one embodiment, the exosome-containing sample can be a sample taken from the body including, but not limited to, blood, urine, mucus, saliva, or tear drops.

After the preparation of the mixture of the sample comprising exosomes and the recombinant exosomes, the method comprises separating the exosomes from the mixture, including the exosomes of the exosome-containing sample and the recombinant exosomes. The separating of the exosomes can be performed using any suitable method, such as a density gradient method, ultracentrifugation, filtration, dialysis, antibody-specific immunoaffinity columns, free-flow electrophoresis (FFE), or a combination thereof.

After the separation of the exosomes, the method includes quantifying the recombinant exosomes in the separated exosomes. The recombinant exosomes allow quantification by detecting fluorescence or luminescence. The quantifying of the recombinant exosomes can be performed using any of a variety of methods which depends on the type of light-emitting protein used in the recombinant exosomes. For example, if the light-emitting protein is a fluorescent protein, the fluorescence of the light-emitting protein when irradiated by ultraviolet (UV) light can be measured using a fluorophotometer. If the light-emitting protein is a luciferase, the intensity of light generated by an ATP-luciferase reaction can be measured using a luminometer.

The method also includes determining an exosome recovery rate from a ratio of the amount of recombinant exosomes after separation to the known amount of recombinant exosomes added to the exosome-containing sample before separation. The ratio of the exosomes after separation to the known amount of the exosomes mixed with the exosome-containing sample can be calculated, and used in calculating the exosome recovery rate. The exosome recovery rate can be used in quantifying exosomal microRNAs or exosomal proteins in the sample, and further can be used in exosome-based diagnosis.

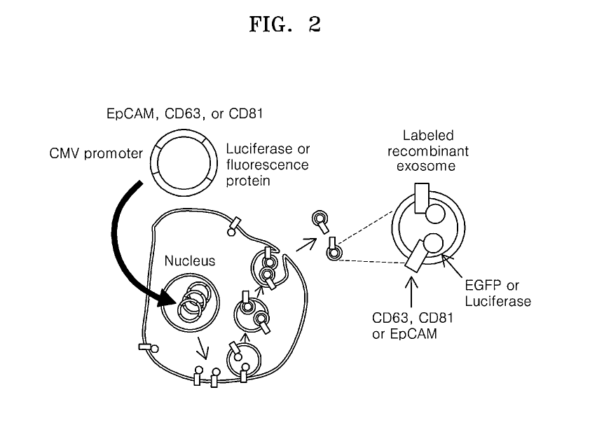

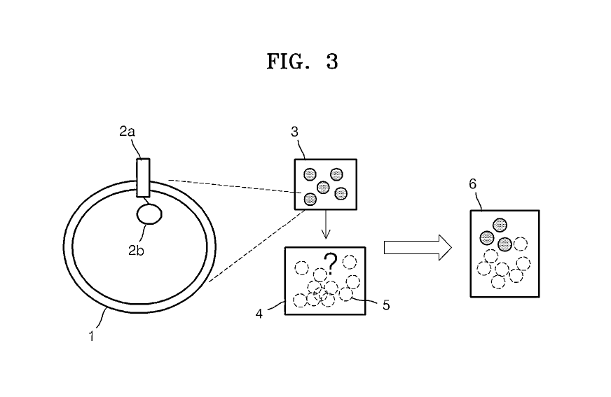

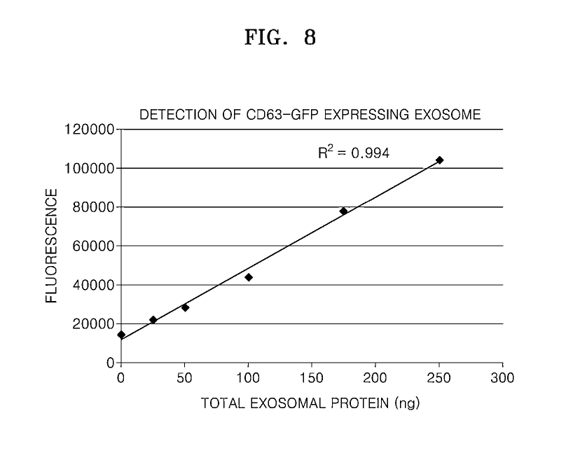

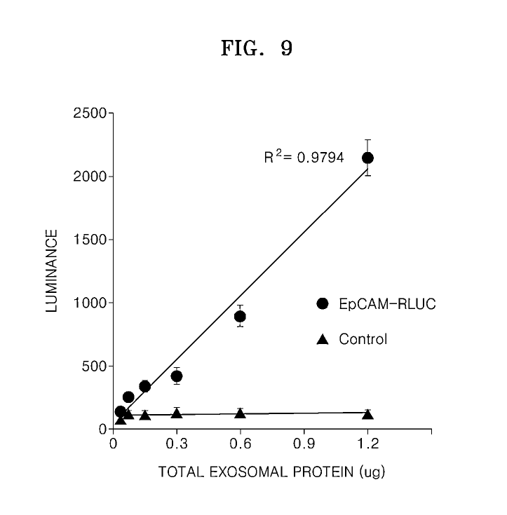

The inventive quantification method enables detection of very small amounts of recombinant exosomes (e.g., about 25 ng of recombinant exosomes as described herein). Proportional increase in fluorescence or luminescence to the total amount of recombinant exosomes in the sample reflects an increase in the exosomal recovery rate; and proportional decrease in the fluorescence or luminescence to the total amount of recombinant exosomes reflects a decrease in exosomal recovery rate. The recovery rate of the recombinant exosomes is representative of the recovery rate of all exosomes in the same, and therefore enables accurate measurement of the overall exosome recovery rate in a given separation (see FIGS. 8 and 9). A method of preparing recombinant exosomes and using the recombinant exosomes to determine exosome recovery rate is illustrated in FIGS. 2 and 3.

The present invention will be described in further detail with reference to the following examples. These examples are for illustrative purposes only and are not intended to limit the scope of the invention.

### EXAMPLE 1

This example demonstrates the vector construction for a fusion protein including a membrane protein and a light-emitting protein.

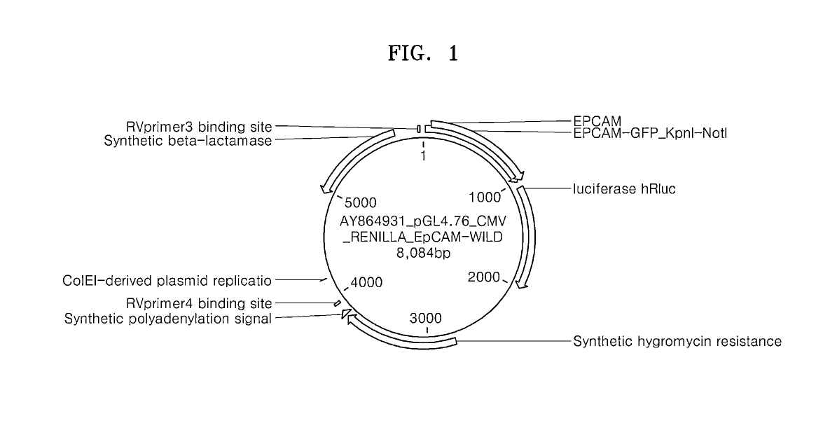

A nucleic acid encoding epithelial cell adhesion molecule (EpCAM) and a nucleic acid encoding a renilla luciferase (RLUC) were inserted into multi-cloning sites (MCS) of pGL4.76 (AY864931) plasmid template with a cytomegalovirus (CMV) promoter. The resulting fusion protein-containing-exosome expression vector is shown in FIG. 1 (see SEQ ID NO: 1).

Vectors encoding fusion proteins of the combinations of membrane protein and light-emitting protein shown in Table 1 were constructed in the same manner as described above.

TABLE 1

| Membrane | | |
|---|---|---|
| protein | Light-emitting protein | SEQUENCE ID No. |
| EpCAM | Luciferase | EpCAM-RLUC |
| | | (SEQ ID NO: 2) |
| CD63 | Luciferase | CD63-RLUC |
| | | (SEQ ID NO: 3) |
| CD81 | Luciferase | CD81-RLUC |
| | | (SEQ ID NO: 4) |
| EpCAM | GFP | EpCAM-GFP |
| | | (SEQ ID NO: 5) |
| CD63 | GFP | CD63-GFP (SEQ ID NO: 6) |
| L1 | GFP | lamp1(L1) (SEQ ID NO: 7) |

### EXAMPLE 2

This example demonstrates the preparation of a recombinant exosome.

### EXAMPLE 2-1

### Introduction of Gene that Encodes the Fusion Protein Including Membrane Protein and Light-Emitting Protein into a Cell Line

Cells were uniformly inoculated on a 150-mm plate and incubated one day before transfection. 7.5 μg of the plasmid vector was diluted in 7.5 ml of an opti-MEM serum-free medium (available from Invitrogen, Grand Island, N.Y.) and thoroughly mixed, followed by an addition of a Plus reagent (available from Invitrogen), a gentle slow mixing, and incubation at room temperature for about 5 minutes. The incubated mixed solution was further gently mixed, and 187.5 μl of Lipofectamine™ reagent (available from Invitrogen) was directly added thereto, thoroughly mixed together, and incubated at room temperature for about 30 minutes to obtain a DNA-lipid complex. The DNA-lipid complex was then slowly added dropwise onto a plate containing MCF-7 cells (ATCC) to be transfected, and mixed with the cells by gentle shaking. The plate with the mixed DNA-lipid complex and cells was incubated in a 37° C. in a CO₂ incubator for about 12-14 hours, followed by an exchange of the culture medium (containing fetal bovine serum (FBS)) with fresh medium (containing FBS but free of exosomes). The cells were incubated in a CO₂ incubator at about 37° C. for about 24-48 hours, and the culture medium was collected.

### EXAMPLE 2

### Isolation of Recombinant Exosomes from the Cell Line

50 μl of the culture medium was transferred into a centrifugation tube, which was then centrifuged at about 300×g at about 4° C. for about 10 minutes. After removal of the supernatant using a pipette, the rest of the centrifuged product was transferred into a new centrifugation tube, which was centrifuged again at about 300×g at about 4° C. for about 10 minutes. After removal of the supernatant using a pipette, the rest of the centrifuged product was transferred to a new centrifugation tube, which was centrifuged again at about 2,000×g at about 4° C. for about 20 minutes. The supernatant was transferred into a clean, empty polyallomer tube or polycarbonate bottom durable against ultracentrifugation, which was centrifuged again at about 10,000×g at about 4° C. for about 30 minutes. The supernatant was transferred into an empty ultracentrifugation tube, which was centrifuged again at about 110,000×g at about 4° C. for about 70 minutes, followed by removal of the supernatant using a pipette. The remaining pellet in the centrifugation tube was re-suspended using 1,000 μl of phosphate buffered saline (PBS). After filling the centrifugation tube with PBS, the centrifugation tube was centrifuged at about 100,000×g at about 4° C. for about 70 minutes, followed by removal of the supernatant as completely as possible.

Re-suspension of the remaining pellet in the centrifugation tube with PBS was followed by centrifugation at about 100,000×g at about 4° C. for about 70 minutes, and removal of the supernatant was done as completely as possible. The remaining pellet was re-suspended by an addition of a small amount of PBS or tris-buffered saline (TBS). The suspension was portioned by about 100 μl, stored at about −80° C., and thawed immediately before use.

### EXAMPLE 3

This example demonstrates the identification of the expression of the light-emitting protein in the recombinant exosome.

### EXAMPLE 3-1

### Targeting Efficiency Depends on Targeting Sequences and the Identification of Light-Emitting Protein's Location in Exosome

After expression of a light-emitting protein in MCF-7 cells as a fusion protein with an exosomal membrane protein, exosomes were isolated from the cells by ultracentrifugation. After lysis of the isolated exosomes, the activity of each luciferase in the exosomes was measured using a Luciferase assay system (Cat No. E2520, available from Promega, Madison, Wis.). The cells in the culture plate were reacted with 100 μl of a luciferase reagent (Steady-Glo Reagent) for about 5 minutes, each sample was transferred to a 96-well plate, and fluorescence in the samples was measured by a fluorescence detector (Luminometer).

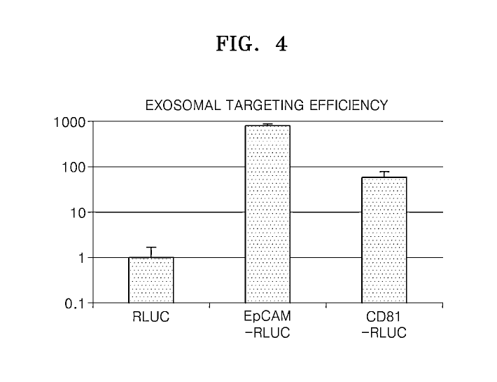

Through analysis of the fluorescence of the samples, an insertion efficiency of the fusion protein into exosomes was measured. The insertion efficiency depends on the presence or types of exosomal targeting sequences in the fusion protein. In particular, an EpCAM-luciferase was found to have an exosomal targeting efficiency that is about 800-fold higher than that of an EpCAM-lacking luciferase. A CD81-luciferase was found to have an about 60-fold higher targeting efficiency compared with a CD81-lacking luciferase (see FIG. 4). These results indicate that EpCAM most efficiently targeted the fusion protein (EpCAM-luciferase) into exosomes.

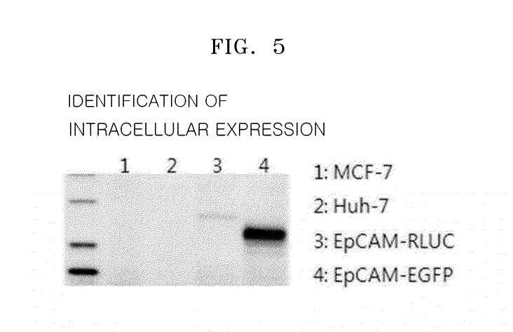

Expression of the fusion protein including the light-emitting protein and membrane protein in the cells was identified using anti-EpCAM antibody-specific western blotting (see FIG. 5).

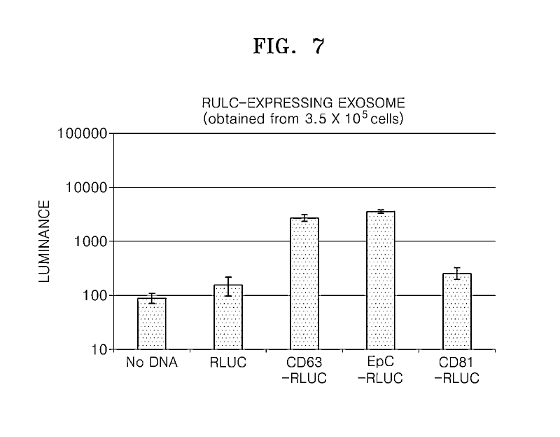

To identify the degrees of expression of the fusion proteins in the cells and exosomal targeting efficiencies according to types of membrane proteins, after over-expression of the fusion proteins including RLUC, EpCAM-RLUC, CD63-RLUC, or CD81-RLUC, luminance from the resulting exosomes in each sample was measured. As a result, the EpCAM-RLUC fusion protein and the CD63-RLUC fusion protein were found to be higher in degree of expression and targeting efficiency than the RLUC protein or the CD81-RLUC fusion protein (see FIG. 7).

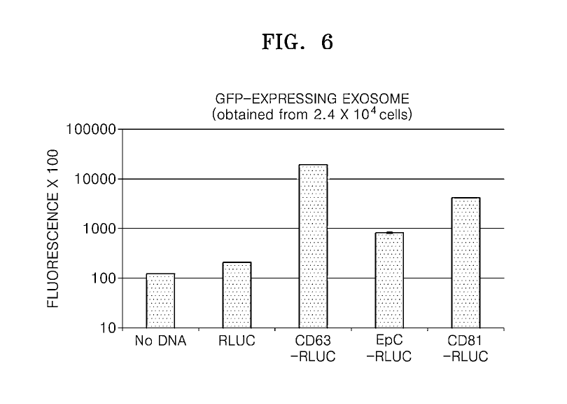

To further identify the degrees of expression of the fusion proteins in the cells and exosomal targeting efficiencies according to types of membrane proteins, after over-expression of the fusion proteins including GFP, CD63-GFP, EpCAM-GFP, or L1-GFP in the same manner as above, the fluorescence from the resulting exosomes in each sample was measured. As a result, the CD63-GFP fusion protein and L1-GFP fusion protein were found to be significantly higher in degree of expression and targeting efficiency than the GFP protein. The EpCAM-GFP fusion protein was higher in degree of expression and targeting efficiency than the GFP protein (see FIG. 6).

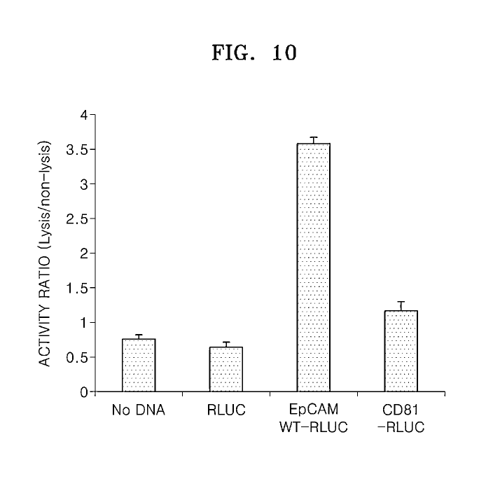

Luminance measurements before and after exosome lysis found that the photoprotein is present in exosomes (see FIG. 10). 78% or greater of the total luminance is attributable to the lysis when EpCAM was used, which indicates the presence of about 78% or greater of the light-emitting protein inside the exosomes (see FIG. 10).

### EXAMPLE 3-2

### Measurement of Minimum Detectable Amount of Exosomes

To identify whether the recombinant exosome is quantitatively measurable, a minimum detectable amount of EpCAM-RLUC expressing exosomes was measured using a Luciferase assay system (Cat No. E2520, available from Promega). As a result of luminance measurement using the luciferase in exosomes, the luminance was found to increase with an increasing amount of exosomes, and a minimum detectable amount of the EpCAM-RLUC expressing exosomes was found to be about 80 ng (see FIG. 7).

A minimum detectable amount of CD63-GFP expressing exosomes was measured using a fluorophotometer. As a result of fluorescent measurement using the GFP in exosomes, the fluorescence was found to increase with an increasing amount of exosomes. The minimum detectable amount of the CD63-GFP expressing exosomes was found to be about 25 ng (see FIG. 8), which is significantly small compared with general quantification of exosomes by Western blotting using only CD63, CD9, or CD81 in which at least about 1-5 μg of exosomes is required to be detected.

### EXAMPLE 4

This example demonstrates the measurement of the exosome recovery rate using recombinant exosomes.

Exosome recovery rates in various types of samples were measured using recombinant exosomes that contained the EpCAM-RLUC fusion protein. Exosome-free serum samples were prepared, and a portion of the samples were mixed with the recombinant exosomes (1.5 μg) to form a mixture.

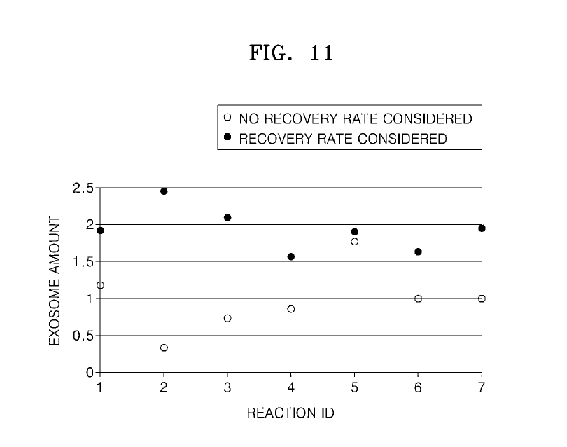

The exosomes then were isolated from the mixture using ultracentrifugation in the same manner as described in Example 2-2. The recombinant exosomes remaining in the samples after the ultracentrifugation were quantified, followed by calculation of exosome recovery rates therefrom. The results are presented in FIG. 11.

As a result, the amounts of the exosomes in the samples were measured to be different in different recovery conditions even though the samples had the same amount of the actual exosomes. However, the average amounts of the exosomes on which the exosome recovery rates were reflected were similar to the actual amounts of the exosomes added to the samples, and had a reduced coefficient of variation (CV) (see Table 2).

TABLE 2

| | Exosome amount | Exosome amount |
|---|---|---|
| | (no recovery rate reflected) | (recovery rate reflected) |
| CV | 1.55 ± 0.24 | 0.79 ± 0.35 |

As described above, according one or more of the embodiments of the present disclosure, a method of exosome recovery rate quantification using a recombinant exosome that includes a fusion protein of a membrane protein and a light-emitting protein enables accurate quantification of the amount of exosomes in a sample, and the amounts of microRNA and protein in the exosomes. Not using antibodies in the quantification of exosomes facilitates the quantification process itself, and the use of a fluorescent protein or luciferase with high sensitivity ensures accurate exosome quantification.

Use of the inventive exosome recovery rate quantification ensures accurate quantification of intracellular exosomes, and thus, improves the efficiency of exosome-based diagnosis.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of determining an exosome recovery rate, the method comprising:
(a) mixing (i) a sample comprising exosomes with (ii) a known amount of recombinant exosomes, wherein the recombinant exosomes comprise a fusion protein of a membrane protein and a light-emitting protein to obtain a mixture;
(b) isolating the exosomes from the mixture;
(c) detecting the amount of the recombinant exosomes among the isolated exosomes; and
(d) determining the exosome recovery rate based on a ratio of the amount of recombinant exosomes after the separation to the known amount of recombinant exosomes mixed with the sample before the separation.

2. The method of claim 1, wherein the membrane protein is an exosomal membrane protein.

3. The method of claim 2, wherein the membrane protein comprises EpCAM, CD63, CD81, or L1.

4. The method of claim 2, wherein the membrane protein comprises an N-terminus of EpCAM.

5. The method of claim 1, wherein the light-emitting protein is a fluorescent protein or a luciferase.

6. The method of claim 1, wherein the light emitting protein is green fluorescent protein (GFP), yellow fluorescent protein (YFP), or red fluorescent protein (RFP).

7. The method of claim 1, wherein the fusion protein includes a membrane protein and a light-emitting protein that are directly linked to each other.

8. The method of claim 1, wherein the fusion protein includes a membrane protein and a light-emitting protein that are linked to each other via a linker.

9. The method of claim 8, wherein the linker comprises about 1 to about 50 amino acids.

10. The method of claim 9, wherein the linker comprises about 5 to about 20 amino acids.

11. The method of claim 1, wherein the light-emitting protein is linked to a C-terminus of the membrane protein and located inside of the exosome.

12. The method of claim 1, wherein the light-emitting protein is linked to an N-terminus of the membrane protein and located outside the exosome.

13. The method of claim 1, wherein the exosomes are isolated using a density gradient method, ultracentrifugation, filtration, dialysis, free-flow electrophoresis, or combination thereof.

14. The method of claim 1, wherein the sample comprises blood, plasma, saliva, or tear drops.

15. A method of preparing a recombinant exosome comprising (a) administering an expression vector to a cell, wherein the expression vector encodes a fusion protein comprising a membrane protein and a light-emitting protein, and (b) isolating the recombinant exosome from the cell.

16. The method of claim 15, wherein the membrane protein is an exosomal membrane protein.

17. The method of claim 16, wherein the membrane protein comprises EpCAM, CD63, CD81, and L1.

18. The method of claim 16, wherein the membrane protein comprises an N-terminus of EpCAM.

19. The method of claim 15, wherein the light-emitting protein is a fluorescent protein or a luciferase.

20. The method of claim 15, wherein the light-emitting protein is green fluorescent protein (GFP), yellow fluorescent protein (YFP), and red fluorescent protein (RFP).


SEQUENCE LISTING (7 sequences)

SEQ ID NO: 1 - DNA, 6084 bp, Artificial
  pGL4.76_CMV_EpCAM_Luciferase sequence
ggcctaactggccggtacctgagctcgctagcctcgaggatatcaagatctgccgccgcgatcgccatggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccatgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcaagatctggcctcggcggccaagcttggcaatccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 2 - DNA, 1932 bp, Artificial
  DNA sequence coding EpCAM-RLUC
atggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccatgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcaagatctggcctcggcggccaagcttggcaatccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaa

SEQ ID NO: 3 - DNA, 1686 bp, Artificial
  DNA sequence coding CD63-RLUC
atggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatgaagcttggcaactccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaa

SEQ ID NO: 4 - DNA, 1698 bp, Artificial
  DNA sequence coding CD81-RLUC
atgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtacagatctggcctcggcggccaagcttggcaatccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaa

SEQ ID NO: 5 - DNA, 1662 bp, Artificial
  DNA sequence coding EpCAM-GFP
atggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccatgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcaacgcggccgctcgagatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagagtttaa

SEQ ID NO: 6 - DNA, 1437 bp, Artificial
  DNA sequence coding CD63-GFP
atggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatgacgcgtacgcggccgctcgagatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagagtt

SEQ ID NO: 7 - DNA, 1254 bp, Artificial
  DNA sequence coding Lamp1(L1)
atggcggcccccggcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacctttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagcaggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcatgctgatccccatcgctgtgggtggtgccctggcggggctggtcctcatcgtcctcatcgcctacctcgtcggcaggaagaggagtcacgcaggctaccagactatctag